(19) United States
(12) Patent Application Publication
(10) Pub. No.: US 2005/0197503 A1
(43) Pub. Date: Sep. 8, 2005

(54) Process for the preparation of N-alkyl-N-methyl-3-hydroxy-3-(2-thienyl)-propylamines

(75) Inventors: Robert Schiffers, Gau-Algesheim (DE); Paul Kreye, Ingelheim (DE); Wolfgang Baumgarten, Gau-Algesheim (DE); Rosemarie Collet, Bad Kreuznach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/060,510

(22) Filed: Feb. 16, 2005

(30) Foreign Application Priority Data
Mar. 5, 2004 (EP) 04 005 272
Jul. 6, 2004 (DE) 10 2004 032 828

(51) Int. Cl. C07D 333/12

(52) U.S. Cl. 549/75

(57) ABSTRACT

The present invention relates to an improved process for preparing chiral N-substituted N-methyl-3-hydroxy-3-(2-thienyl)-propylamine on an industrial scale using an asymmetric hydrogenation as a key step and optionally a special sequence of subsequent steps, using a catalyst system consisting of rhodium and (2R, 4R)-4-(dicyclohexylphosphino)-2-(diphenyl-phosphino-methyl)-N-methyl-aminocarbonyl-pyrrolidine.

### RELATED APPLICATIONS

This application claims priority to European Application No. 04 005 272.2, filed on Mar. 5, 2004, and also claims priority to German Application No. 10 2004 032 828.5, filed on Jul. 6, 2004, each of which is hereby incorporated by reference in its entirety.

### FIELD OF THE INVENTION

The present invention relates to an improved process for preparing an (S)—N-substituted-N-methyl-3-hydroxy-3-(2-thienyl)-propylamine by rhodium-catalysed asymmetric hydrogenation on an industrial scale.

### TECHNOLOGICAL BACKGROUND TO THE INVENTION

(S)—N-alkyl-N-methyl-3-hydroxy-3-(2-thienyl)-propylamines are valuable intermediate products for the synthesis of the pharmaceutical active substance duloxetine or (S)—N-methyl-3-(1-naphthyloxy)-3-thienylpropylamine, which belongs to the norepinephrine and serotonin uptake inhibitors used pharmaceutically as antidepressants or agents for treating urinary incontinence and is of great commercial interest. The chemical structure of a chiral (S)—N-alkyl-N-methyl-3-hydroxy-3-(2-thienyl)-propylamine is shown in formula I: 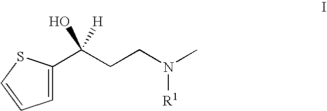
where R¹ denotes a —C₁₋₆-alkyl group optionally substituted by one or more phenyl groups.

The processes for preparing duloxetine known from the prior art include the reaction of 2-acetylthiophene with dimethylamine and formaldehyde in a Mannich reaction, to obtain 3-dimethylamino-1-(2-thienyl)-propanone, subsequent reduction, reaction with 1 fluoronaphthalene and racemate cleaving with optically active acids or chromatography on a chiral stationary phase according to EP 0 273 658; or by asymmetric reduction with lithium aluminium hydride in the presence of a chiral ligand [(2R,2S)−(−)4-dimethylamino-1,2-diphenyl-3-methyl-2-butanol] to form the optically active alcohol and subsequent reaction with 1-fluoronaphthalene according to EP 0 457 559.

Moreover, International Patent Application WO 03/070720 proposes the conversion of 3-N-benzyl-N-methylamino-1-(2-thienyl)-propanone into a corresponding N-alkoxycarbonyl-N-methylamino-1-(2-thienyl)-propanone and subsequent enantioselective reduction thereof, for example using a chiral oxazaborolidine catalyst.

In addition, Ohkuma et al. describe the enantioselective hydrogenation of 3-dimethylamino-1-(2-thienyl)-propanone using a chiral ruthenium catalyst in the presence of potassium tert-butoxide (T. Ohkuma et al. Organic Letters 2000, Vol. 2 No. 12 1749-1751).

International Patent Application WO 2004/011452 proposes the enantioselective hydrogenation of substituted 3-amino-1-(2-thienyl)-propanones using chiral ruthenium catalysts in the presence of diamines.

However, the processes previously described are less suitable for the preparation of (S)—N-substituted N-methyl-3-hydroxy-3-(2-thienyl)-propylamines on an industrial scale, as either the optical purities obtained are unsatisfactory or large amounts of chiral reduction systems have to be used for the enantioselective reduction which are difficult to obtain and in some cases unstable.

### SUMMARY OF THE INVENTION

One of the essential objectives of the present invention is to provide a process by which (S)—N-alkyl-N-methyl-3-hydroxy-3-(2-thienyl)-propylamines of formula I may be prepared with high optical and chemical purity. In this way the risk of contamination of the drug duloxetine with the unwanted (R)-enantiomer is minimised.

A further aim of the invention is to provide a process by which substantially enantiomerically pure (S)—N-alkyl-N-methyl-3-hydroxy-3-(2-thienyl)-propylamine of formula I may be prepared in a simple manner starting from easily obtainable starting materials.

Surprisingly it has now been found that (S)—N-alkyl-N-methyl-3-hydroxy-3-(2-thienyl)-propylamines of formula I may be obtained on an industrial scale in good yields and with very good optical purity if a corresponding N-alkyl-N-methylamino-1-(2-thienyl)-propanone of formula II is subjected to asymmetric hydrogenation in the presence of rhodium and a chiral bidentate phosphine ligand as catalyst system in the absence of a diamine.

### DESCRIPTION OF THE INVENTION

The present invention relates to a process for preparing chiral N-alkyl-N-methyl-3-hydroxy-3-(2-thienyl)-propylamines, of formula I, 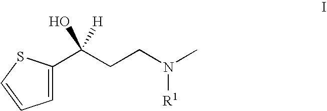
wherein R¹ denotes a C₁₋₆-alkyl group optionally substituted by one or more phenyl groups, or an acid addition salt thereof, starting from prochiral 1-(N-alkyl-N-methylamino)-3-(2-thienyl)-propan-3-one of formula II, 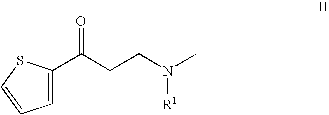
wherein R¹ is as hereinbefore defined, or an acid addition salt thereof, characterised in that the compound of formula II is subjected to asymmetric hydrogenation in the presence of a catalyst system consisting of rhodium, (2R, 4R)-4-(dicyclohexylphosphino)-2-(diphenylphosphino-methyl)-N-methyl-aminocarbonyl-pyrrolidine, optionally an inert diluent and a weak base, preferably a tertiary amine, an alkali metal hydrogen carbonate, alkali metal carbonate or the free base 1-(N-alkyl-N-methylamino)-3-(2-thienyl)-propan-3-one.

By the term “C₁₋₆-alkyl” (including those which are part of other groups) are meant branched and unbranched alkyl groups with 1 to 6 carbon atoms, and accordingly by the term “C₁₋₄-alkyl” are meant branched and unbranched alkyl groups with 1 to 4 carbon atoms. Alkyl groups with 1 to 4 carbon atoms are preferred. Examples of these include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. may optionally also be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl is includes iso-butyl, sec-butyl and tert-butyl etc.

By the term “optionally substituted by one or more phenyl groups” are meant branched and unbranched alkyl groups, wherein one or more, preferably one, two or three hydrogen atoms on one or more adjacent or non-adjacent carbon atoms are replaced by a phenyl group.

The above process wherein R¹ denotes methyl, ethyl, iso-propyl, tert-butyl, benzyl, 1-phenylethyl, 2-phenylethyl, diphenylmethyl or trityl, particularly methyl or benzyl, is preferred.

The above process is particularly preferred for preparing chiral N-N-dimethyl-3-hydroxy-3-(2-thienyl)-propylamine or an acid addition salt thereof starting from prochiral 1-(N-N-dimethylamino)-3-(2-thienyl)-propan-3-one or from chiral N-benzyl-N-methyl-3-hydroxy-3-(2-thienyl)-propylamine or an acid addition salt thereof starting from prochiral 1-(N-benzyl-N-methylamino)-3-(2-thienyl)-propan-3-one or an acid addition salt thereof, particularly the hydrochloride.

In a preferred process, the asymmetric hydrogenation is carried out in a temperature range from 0° C. to 100° C., preferably from 0° C. to 50° C., more preferably from 20° C. to 40° C.

Also preferred is a process wherein the asymmetric hydrogenation is carried out under a pressure of more than 1 bar to 200 bar, preferably under a pressure of from 10 bar to 150 bar, more preferably at 40 to 120 bar.

The inert diluents used may be both protic solvents—such as e.g. alcohols and/or water or aprotic polar solvents such as e.g. ethers and/or amides or lactams and/or mixtures thereof. Water may optionally be added to all the solvents. The protic solvents used are preferably branched or unbranched C₁-C₈ alkanols.

By the term “C₁₋₈-alcohol” are meant branched and unbranched alcohols with 1 to 8 carbon atoms and one or two hydroxy groups. Accordingly, by the term “C₁₋₄alcohols” are meant branched and unbranched alcohols with 1 to 4 carbon atoms and one or two hydroxy groups. Alcohols with 1 to 4 carbon atoms are preferred. Examples of these include: methanol, ethanol, n-propanol, iso-propanol, n-butanol, iso-butanol, sec-butanol, tert-butanol, n-pentanol, iso-pentanol, neo-pentanol or hexanol. The abbreviations MeOH, EtOH, n-PrOH, i-PrOH, n-BuOH, i-BuOH, t-BuOH, etc. may optionally be used for the above-mentioned molecules. Unless otherwise stated, the definitions propanol, butanol, pentanol and hexanol include all the possible isomeric forms of the groups in question. Thus, for example, propanol includes n-propanol and iso-propanol, butanol includes iso-butanol, sec-butanol and tert-butanol etc.

Particularly preferably, lower alcohols such as methanol, ethanol, n-propanol and isopropanol or mixtures thereof are used. Methanol is particularly preferably used as the reaction medium, while the methanol or the other alcohols or solvents may optionally contain water. Suitable aprotic solvents are polar ethers such as for example tetrahydrofuran or dimethoxyethylether or amides such as for example dimethylformamide, or lactams such as for example N-methylpyrrolidone. Preferably, solvents with only a low tendency to flammability are used.

The enantioselective hydrogenation is carried out in the absence of a diamine.

The reaction is preferably carried out in the presence of a weak base. The base used may be an organic base or an inorganic base both in solid form and also in the form of solutions, e.g. aqueous solutions. Suitable inorganic bases are basically reacting alkali metal salts or alkali metal hydroxides. Preferably, alkali metal hydrogen carbonates or alkali metal carbonates are used in addition to alkali metal hydroxides. Most preferably, Na₂CO₃, K₂CO₃, LiOH, NaOH, KOH or NaHCO₃ is used.

Suitable organic bases are tertiary amines, particularly tertiary alkyl-amines, tertiary alkyl-aryl-amines or pyridines or the free base 1-(N-alkyl-N-methylamino)-3-(2-thienyl)-propan-3-one present in excess. Preferably trialkylamines with branched or unbranched C₁-C₆-alkyl groups are used. Triethylamine or diisopropylethylamine have proved particularly preferable for example. If desired, the reaction may also be carried out in the presence of basic polymers with e.g. tertiary amino functions.

Preferred methods are those wherein 1-(N-alkyl-N-methylamino)-3-(2-thienyl)-propan-3-one or the acid addition salt thereof is used in a molar ratio to the rhodium catalyst of from 500:1 to 100000:1, preferably from 750:1 to 20000:1 during asymmetric hydrogenation.

With a molar ratio of catalyst to substrate of about 1:2000, (S)—N-alkyl-N-methyl-3-hydroxy-3-(2-thienyl)-propylamine is obtained with an optical purity of≧94% ee by the process according to the invention starting from 1-(N-alkyl-N-methylamino)-3-(2-thienyl)-propan-3-one hydrochloride.

By reducing the amount of catalyst and using the commercially favourable 1-(N-alkyl-N-methylamino)-3-(2-thienyl)-propan-3-one hydrochloride as educt, the costs of producing (S)—N-alkyl-N-methyl-3-hydroxy-3-(2-thienyl)-propylamine and hence duloxetine may be reduced significantly by the new process.

The 1-(N-alkyl-N-methylamino)-3-(2-thienyl)-propan-3-one to be used as starting material is obtained by reacting 2-acetylthiophene with a corresponding N-alkyl-N-methylamine and formaldehyde in a Mannich reaction.

In addition, the space-time yield can be improved over that of the prior art using the new process. It is particularly advantageous for preparing (S)—N-alkyl-N-methyl-3-hydroxy-3-(2-thienyl)-propylamines on an industrial scale from the point of view of costs and safety.

According to the invention the catalyst used is [Rh(COD)Cl]₂, where COD denotes a cyclooctadienyl group, and (2R, 4R)-4-(dicyclohexylphosphino)-2-(diphenylphosphino-methyl)-N-methyl-aminocarbonylpyrrolidine (RR-MCCPM) as a chiral, bidentate phosphine ligand (PP*).

The preparation of this catalyst is known from the prior art [EP-A-0 251 164, EP-A-0 336 123]. The catalyst may also be bound to the polymer, e.g. by having the chiral ligand (2R, 4R)-4-dicyclohexylphosphino)-2-(diphenylphosphino-methyl)-N-methyl-aminocarbonyl) pyrrolidine bound to a polymer via the phenyl groups, for example. The use of such polymer-bound ligands does not totally rule out the simultaneous use of non-polymer-bound ligands. Such polymer-bound catalysts are particularly advantageous for simple purification of the product.

The catalyst is either used as a prefabricated, oxygen-free solution of [Rh(COD)Cl]₂ and ligand or prepared in situ from [Rh(COD)Cl]₂ and ligand in the presence of 1-(N-alkyl-N-methylamino)-3-(2-thienyl)-propan-3-one without oxygen in a protective gas atmosphere or hydrogen atmosphere.

The hydrogenation is generally carried out without oxygen, conveniently under inert gas, preferably under a hydrogen atmosphere. However, it is not essential to the reaction that the hydrogen for the hydrogenation should be capable of being taken from the atmospheric gas above the reaction mixture. The hydrogen may also be produced in solution in situ from suitable hydrogen sources. Such hydrogen sources include e.g. ammonium formate, formic acid and other formates, hydrazines in the presence of metal ions such as Fe²⁺/Fe³⁺ and other hydrogen sources known from the prior art.

The reaction time for the asymmetric hydrogenation to be completed is generally between 2 and 48 hours, preferably between 4 and 36 hours, and particularly preferably about 18 to 24 hours.

The reaction may be worked up in the conventional manner, for example, by optionally deactivating and separating off the catalyst, removing the solvent from the residue and isolating the pure end product by crystallisation, distillation, extraction or chromatography.

Preferably the following steps are carried out for working up and isolating the product:
(i) dividing the reaction mixture obtained in the asymmetric hydrogenation between water and an organic solvent,
(ii) adjusting the pH of the aqueous phase to a value of from 0.51 to 2,
(iii) separating off the aqueous phase,
(iv) optionally repeating steps (i) to (iii)
(v) adjusting the pH of the aqueous phase to 5.5 to 10
(vi) dividing the reaction mixture between water and an organic solvent,
(vii) optionally repeating steps (v) to (vi)
(vii) separating off the organic phase formed and concentrating.

In particular, for working up and isolating the product after enantioselective hydrogenation, the reaction mixture obtained is evaporated down and the solid obtained is divided between water and an organic solvent, particularly toluene or dichloromethane. The pH of the aqueous phase is adjusted to a value of 0 to 2, preferably 0.05 to 1.8, particularly 0.1 to 1.6, then the aqueous phase is separated off. The organic phase is preferably combined again with water, acidified, and separated off again. The combined aqueous phases are adjusted to a pH of 5.5 to 10, preferably 6.0 to 9.5, particularly 6.4 to 9, combined with solvent and extracted. The N-alkyl-N-methyl-3-hydroxy-3-(2-thienyl)-propylamine is obtained after elimination of the solvent as a solid of high chemical and optical purity.

The enantiomeric purity can be further increased by recrystallisation from a suitable solvent.

It is possible to increase the enantiomeric excess to >99% for the product by recrystallisation from a non-polar solvent such as n-pentane, n-heptane or cyclohexane, particularly n-heptane. The same is true of a corresponding acid addition salt, such as for example the oxalate or mandelate. However, in this case, the product is recrystallised from a polar solvent such as methanol, ethanol or isopropanol, or a mixture of isopropanol and toluene.

The product obtained is converted into duloxetine in a manner known per se either by (a) reacting with 1-fluoronaphthalene and subsequently cleaving the alkyl group R¹ or by (b) cleaving the alkyl group R¹ and subsequently reacting with 1-fluoronaphthalene.

The process according to the invention is illustrated by the following Examples. The skilled person will be aware that the Examples are intended only as an illustration and are not to be regarded as limiting.

### EXAMPLES

### Example 1

### Preparation of N-benzyl-N-methylamine-hydrochloride

545 g (4.5 mol) of N-benzyl-methylamine are taken up in 1600 ml of toluene and 536 g (4.7 mol) of 32% hydrochloric acid are carefully added, with stirring, while the mixture is heated to about 80° C. Then it is refluxed using the water separator. After about 4 hours, approximately 300 ml of water are separated off, whereupon crystals are precipitated. Another 200 ml of toluene are added and water is separated off again while refluxing. After all the water has been removed the suspension obtained is cooled to ambient temperature. After adding 500 ml of acetone, the mixture is cooled to about 10° C. and the crystals are separated off and washed with acetone. The damp crystals thus obtained are dried, yield 695.6 g (98.0% of theory)

### Example 2

### Preparation of 1-(N-benzyl-N-methylamino)-3-(2-thienyl)-propan-3-one-hydrochloride

464.2 g (3.7 mol) 2-acetylthiophene are taken up in 283 ml of ethanol; 110.3 g (3.7 mol) paraformaldehyde are added with stirring, and the mixture is rinsed with 116 ml of ethanol. Then, 579.9 g (3.7 mol) of N-benzyl-N-methylamine-hydrochloride are added, and the suspension formed is refluxed. After about 45 minutes, crystals are precipitated out. After another 15 minutes, the suspension is diluted with 200 ml of ethanol and cooled to about 10° C. The crystals are separated off and washed with cold ethanol in batches. The damp, pure white crystals are dried, yield: 814.4 g (74.8% of theory), purity: 95% (HPLC).

### Example 3

### (S)—N-benzyl-N-methyl-3-hydroxy-3-(2-thienyl)-propylamine

296 g (0.95 mol) 1-(N-benzyl-N-methylamino)-3-(2-thienyl)-propan-3-one-hydrochloride (95%) are suspended in 6.1 litres of methanol under nitrogen; 72 mg of bis-(1,5-cyclooctadiene)-dirhodium(I)-dichloride, 153 mg of (2R, 4R)-4-dicyclohexylphosphino)-2-(diphenylphosphino-methyl)-N-methyl-aminocarbonyl) pyrrolidine (as a toluene solution) and 610 mg sodium hydrogen carbonate are added. The suspension obtained is hydrogenated at 40° C. and 50 bar hydrogen pressure for about 20 hours. Monitoring of the process by HPLC shows >99% reaction, 0.2% educt.

The reaction mixture is evaporated down and the solid obtained is divided between 1.5 L water and 1.5 L of an organic solvent (toluene or dichloromethane). The pH is adjusted to about 0.1 (pH electrode) with 32% hydrochloric acid and the mixture is vigorously stirred for 10 minutes, then the aqueous phase is separated off. The organic phase is again combined with 0.9 L water, adjusted to pH 0.1, stirred and the aqueous phase is separated off again. The combined aqueous phases are then adjusted with 45% sodium hydroxide solution to a pH of precisely 6.4, after which the N-benzyl-N-methyl-3-hydroxy-3-(2-thienyl)-propylamine is precipitated as a clear organic phase and separated off. The aqueous phase remaining is extracted again with 0.9 L solvent and the combined organic phases are evaporated down at 50° C. and 5 mbar. The product is a colourless oil, chemical purity 98.5% (HPLC, 0.2% educt, 0.3% N-benzyl-N-methylamine, 0.2% 2-acetylthiophene), enantiomeric purity 98% (NMR, comparison with racemate).

### Example 4

### Preparation of 1-(N,N-dimethylamino)-3-(2-thienyl)-propan-3-one hydrochloride

252.4 g (2.0 mol) 2-acetylthiophene are dissolved in 160 ml isopropanol and added with stirring to 60.1 g (2.0 mol) of paraformaldehyde. Then, 163.1 g (2.0 mol) dimethylamine-hydrochloride are added and the mixture is rinsed with another 100 ml isopropanol. The thick suspension obtained is refluxed for about 3 hours. The suspension is diluted with another 400 ml isopropanol and cooled to about 15° C., suction filtered and washed with 400 ml isopropanol in batches; then it is dried overnight at 60° C. in the vacuum drying cupboard, yield 265.6 g (60.4% of theory), purity >98% according to NMR.

### Example 5

### N,N-dimethyl-3-hydroxy-3-(2-thienyl)-propylamine

70 g (0.32 mol) of 1-(N,N-dimethylamino)-3-(2-thienyl)-propan-3-one-hydrochloride are suspended in 630 ml of methanol and 70 ml of water under nitrogen; 16.5 mg bis-(1.5-cyclooctadiene)-dirhodium(I)-dichloride, 34.9 mg (2R, 4R)-4-dicyclohexylphosphino)-2-(diphenylphosphino-methyl)-N-methyl-aminocarbonyl) pyrrolidine and 140 mg sodium hydrogen carbonate are added and the suspension is hydrogenated at 30° C. and 100 bar hydrogen pressure for about 20 hours.

Then the reaction mixture is evaporated down and the residue obtained is divided between 350 ml of water and 250 ml organic solvent (toluene or dichloromethane). The pH is adjusted to 1.6 with 32% hydrochloric acid and the mixture is stirred for 10 minutes, then the aqueous phase is separated off. The organic phase is again combined with 250 ml of water, stirred and the aqueous phase is again separated off. The combined aqueous phases are adjusted to pH 9.0 with 400 ml organic solvent and 45% sodium hydroxide solution, stirred, and then the phases are separated. The aqueous phase is extracted again with 200 ml solvent and the combined organic phases are evaporated down at 60° C. and 5 mbar. The yield of crude product is 50.0 g (85% of theory), chemical purity >98% (NMR).

The crude product is recrystallised from 150 ml n-heptane, washed with another 50 ml of n-heptane and dried overnight at 40° C. and 5 mbar. 46.8 g (79% of theory) of S—N, N-dimethyl-3-hydroxy-3-(2-thienyl)-propylamine are obtained as a white solid, purity >98% (NMR), enantiomeric purity 94% (HPLC), melting point 76-78° C.

### Example 6

### First recrystallisation of S—N,N-dimethyl-3-hydroxy-3-(2-thienyl)-propylamine

2.5 g of S—N,N-dimethyl-3-hydroxy-3-(2-thienyl)-propylamine, ee =97% (HPLC), are recrystallised from 7.5 mL n-heptane, washed with another 10 mL n-heptane and dried at 40° C. and 5 mbar. 2.3 g (92%) product are obtained, enantiomeric purity 99.6% (HPLC).

### Example 7

### Second recrystallisation of S—N,N-dimethyl-3-hydroxy-3-(2-thienyl)-propylamine

2.1 g of S—N,N-dimethyl-3-hydroxy-3-(2-thienyl)-propylamine, ee =99,6% (HPLC), are recrystallised from 6.3 mL n-heptane, washed with another 10 mL n-heptane and dried at 40° C. and 5 mbar. 1.9 g (91% of theory ) of product are obtained, enantiomeric purity 100% (HPLC).

1. A process for preparing chiral N-alkyl-N-methyl-3-hydroxy-3-(2-thienyl)-propylamine of formula I, 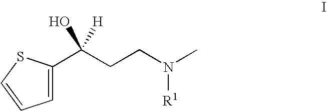
wherein R¹ denotes a —C₁₋₆-alkyl group optionally substituted by phenyl, or an acid addition salt thereof, starting from prochiral 1-(N-alkyl-N-methylamino)-3-(2-thienyl)-propan-3-one of formula II, 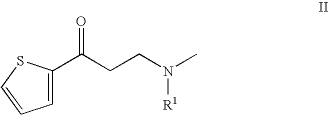
wherein R¹ is as hereinbefore defined, or an acid addition salt thereof, comprising subjecting the compound of formula II to asymmetric hydrogenation in the presence of a catalyst system consisting of rhodium, (2R, 4R)-4-(dicyclohexylphosphino)-2-(diphenylphosphino-methyl)-N-methyl-aminocarbonyl-pyrrolidine and, optionally, an inert diluent and a weak base.

2. The process according to claim 1, wherein the hydrochloride of the 1-(N-alkyl-N-methylamino)-3-(2-thienyl)-propan-3-one of formula II is used as an educt.

3. The process according to claim 1, wherein the hydrogenation is carried out in the presence of less than one equivalent of a weak base selected from the group consisting of tertiary amines, alkali metal hydrogen carbonates, alkali metal-carbonates and the free base 1-(N-alkyl-N-methylamino)-3-(2-thienyl)-propan-3-one of formula II.

4. The process according to claim 1, wherein the asymmetric hydrogenation is carried out in a temperature range of from 0° C. to 100° C.

5. The process according to claim 4, wherein the asymmetric hydrogenation is carried out in a temperature range of from 0° C. to 50° C.

6. The process according to claim 5, wherein the asymmetric hydrogenation is carried out in a temperature range of from 20° C. to 40° C.

7. The process according to claim 1, wherein the asymmetric hydrogenation is carried out under a pressure of from more than 1 bar to 200 bar.

8. The process according to claim 7, wherein the asymmetric hydrogenation is carried out under a pressure of 10 bar to 150 bar.

9. The process according to claim 8, wherein the asymmetric hydrogenation is carried out at 40 bar to 120 bar.

10. The process according to claim 1, wherein the asymmetric hydrogenation is carried out in a protic diluent.

11. The process according to claim 10, wherein the asymmetric hydrogenation is carried out in a branched or unbranched C₁₋₈-alcohol as diluent.

12. The process according to claim 11, wherein the branched or unbranched C₁₋₈-alcohol is selected from the group consisting of methanol, ethanol, n-propanol, and isopropanol.

13. The process according to claim 11, wherein the diluent for the asymmetric hydrogenation contains water.

14. The process according to claim 1, wherein 1-(N-alkyl-N-methylamino)-3-(2-thienyl)-propan-3-one of formula II or an acid addition salt thereof is used in a molar ratio of 500:1 to 100000:1 to the rhodium catalyst in the asymmetric hydrogenation.

15. The process according to claim 14, wherein the molar concentration is from 750:1 to 20000:1.

16. The process according to claim 15, wherein the molar concentration is 2000:1.

17. The process according to claim 14, wherein the rhodium catalyst is used as a pre-prepared solution for the asymmetric hydrogenation.

18. The process according to claim 14, wherein the rhodium catalyst for the asymmetric hydrogenation is produced in situ.

19. The process according to claim 1, wherein the asymmetric hydrogenation is carried out within a reaction time of 2 to 48 hours.

20. The process according to claim 19, wherein the asymmetric hydrogenation is carried out within a reaction time of 4 to 36 hours.

21. The process according to claim 20 wherein the asymmetric hydrogenation is carried out within a reaction time of about 20 hours.

22. The process according to claim 1, further comprising the following steps:
(i) dividing the reaction mixture obtained in the asymmetric hydrogenation between water and an organic solvent;
(ii) adjusting the pH of the aqueous phase to a value of from 0.1 to 2;
(iii) separating off the aqueous phase;
(iv) optionally repeating steps (i) to (iii);
(v) adjusting the pH of the aqueous phase to 5.5 to 10;
(vi) dividing the reaction mixture between water and an organic solvent;
(vii) optionally repeating steps (v) to (vi); and
(vii) separating off the organic phase formed and concentrating,
wherein the product or an acid addition salt thereof is isolated.

23. The process according to claim 22, wherein the isolated product or an acid addition salt thereof is recrystallised from a suitable solvent, in order to increase the enantiomeric purity.

24. A process for preparing duloxetine, comprising reacting the chiral N-alkyl-N-methyl-3-hydroxy-3-(2-thienyl)-propylamine I prepared according to claim 1 with 1-fluoronaphthalene and cleaving the alkyl group R¹.

25. A process for preparing duloxetine, comprising reacting the chiral N-alkyl-N-methyl-3-hydroxy-3-(2-thienyl)-propylamine I prepared according to claim 22 with 1-fluoronaphthalene and cleaving the alkyl group R¹.

26. A process for preparing duloxetine, comprising: (a) cleaving the alkyl group R¹ of the chiral N-alkyl-N-methyl-3-hydroxy-3-(2-thienyl)-propylamine I prepared according to claim 1; and (b) reacting the product obtained in step (a) with 1-fluoronaphthalene.

27. A process for preparing duloxetine, comprising: (a) cleaving the alkyl group R¹ of the chiral N-alkyl-N-methyl-3-hydroxy-3-(2-thienyl)-propylamine I prepared according to claim 22; and (b) reacting the product obtained in step (a) with 1-fluoronaphthalene.
